(19) United States
(12) Patent Application Publication
(10) Pub. No.: US 2008/0287341 A1
(43) Pub. Date: Nov. 20, 2008

(54) TREATMENT OF VASCULAR ABNORMALITIES USING NANOPARTICLES

(76) Inventor: Danyang Chen, Oklahoma City, OK (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 11/750,983

(22) Filed: May 18, 2007

(51) Int. Cl. A61K 38/00; A61P 27/00

(52) U.S. Cl. 514/2

(57) ABSTRACT

Vascular abnormalities in a patient are treated by administering a therapeutically effective amount of an agent that reduces angiogenesis. The agent comprises an vascular leakage angiogenic inhibitor and/or vascular leakage angiogenic inhibitor expression plasmid/vector encapsulated by a biocompatible nanoparticle.

### BACKGROUND

Retinal vascular leakage and neovascularization are the major features of diabetic retinopathy, macular degeneration, and the leading causes of vision loss. These retinal vascular abnormalities are also common in other ocular disorders such as sickle cell retinopathy, retinal vein occlusion and retinopathy of prematurity (ROP). Vascular endothelial growth factor (VEGF) is known to play a key pathogenic role in the blood-retinal barrier breakdown or vascular leakage and retinal neovascularization.

Angiogenesis is regulated by two counter-balancing systems between angiogenic stimulators, such as VEGF, and vascular leakage angiogenic inhibitors, such as angiostatin. Angiostatin contains the first four triple disulfide bond-linked loops of plasminogen, known as kringle domains and is a potent inhibitor of angiogenesis. Among proteolytic fragments of plasminogen, kringle 5 (K5), an 80-amino acid peptide from plasminogen, has the most potent inhibitory effect on endothelial cell growth. K5 has been shown to inhibit ischemia-induced retinal neovascularization in oxygen-induced retinopathy (OIR) models. K5 has also been shown to reduce retinal vascular leakage in OIR models and in streptozotocin-induced diabetes models. The K5 -induced reduction of vascular leakage can be achieved through an intraocular, periocular, topical or systemic administration of the K5 peptide. Similar to many other anti-angiogenic peptides, however, these K5 effects are transient after a single injection of the peptide due to short half-life of the peptide in the retina. A sustained ocular delivery of K5, such as gene therapy, is desirable for development of a long-term treatment of diabetic retinopathy.

Traditionally, gene delivery systems can be classified into viral vector-mediated and non-viral deliveries. Currently, viral vectors are the most commonly used means for gene delivery, due to their high efficiencies. The limitations of viral vector-mediated delivery, such as potential risks, restricted targeting of specific cell types, and immunogenecity of viral vectors hamper their clinical application. For these reasons, non-viral systems for gene delivery have become increasingly desirable in both basic research and clinical settings.

### SUMMARY

In accordance with at least one aspect of the present disclosure, a method is provided for treating vascular abnormalities in a patient. The method comprises administering to the patient a therapeutically effective amount of an agent that reduces vascular leakage and angiogenesis. The agent is provided as biocompatible nanoparticles that contain an vascular leakage angiogenic inhibitor and/or an vascular leakage angiogenic inhibitor expression plasmid/vector.

According to embodiments of the instant teachings, the method for treating vascular abnormalities is a sustained therapeutic effect.

In accordance with other aspects of the present disclosure, a method is provided for preparing an agent for treating vascular abnormalities. The method comprises encapsulating an vascular leakage angiogenic inhibitor and/or vascular leakage angiogenic inhibitor expression plasmid/vector in biocompatible nanoparticles.

In at least one embodiment, encapsulating an vascular leakage angiogenic inhibitor and/or vascular leakage angiogenic inhibitor expression plasmid/vector comprises dissolving the polymer in a solvent, mixing the vascular leakage angiogenic inhibitor or vascular leakage angiogenic inhibitor expression plasmid/vector with the polymer, emulsifying the mixture, and evaporating the solvent.

In accordance with further aspects of the present disclosure, an agent is provided for treating vascular abnormalities. The agent comprises biocompatible nanoparticles containing an vascular leakage angiogenic inhibitor and/or vascular leakage angiogenic inhibitor expression plasmid/vector.

In other and further embodiments, the vascular leakage angiogenic inhibitor is kringle 5 and the nanoparticle is formulated from PLGA.

### DRAWINGS

The above-mentioned features and objects of the present disclosure will become more apparent with reference to the following description taken in conjunction with the accompanying drawings wherein like reference numerals denote like elements and in which:

FIG. 1 is a collection of images showing K5 expression from K5-NP in vitro, according to the teachings of the instant disclosure;

FIG. 2 is a bar graph illustrating the effect of K5-NP on ARPE19 cells and BRCEC growth, according to the teachings of the instant disclosure;

FIG. 3 is a bar graph illustrating the effect of K5-NP on VEGF over-expression under hypoxia, according to the teachings of the instant disclosure:

FIG. 4 is a collection of images showing K5 expression in the rat retina after an intravitreal injection of K5-NP, according to the teachings of the instant disclosure.

FIG. 5 is a bar graph illustrating the effect of K5-NP on retinal vascular permeability in OIR rats; according to the teachings of the instant disclosure; and

FIG. 6 is a collection of images and graphs showing the effect of K5-NP on retinal neovascularization in OIR rats after intravitreal injection of K5; according to the teachings of the instant disclosure.

### DETAILED DESCRIPTION

As used above and elsewhere herein the following terms and abbreviations have the meanings defined below:

AMD (ARMD) Age-related macular degeneration

bFGF Basic fibroblast growth factor

BRB Blood-retina barrier

DME Diabetic macular edema

DR Diabetic retinopathy

ERG Electroretinogram

GFAP Glial fibrillary acid protein

GLP Good laboratory procedures

GMP Good manufacturing procedure

HIF-1 Hypoxia induced factor-1

IGF-1 Insulin-like growth factor 1

IND Investigational new drug

K5 Plasminogen kringle 5

K5-NP K5 nanoparticles

MTT 3-(4,5-dimethylthiazol-2-yl)-2,5-diphenyl tetrazolium bromide

NP Nanoparticles

NV Neovascularization

OIR Oxygen-induced retinopathy

PDR Proliferative diabetic retinopathy

PEDF Pigment epithelium-derived factor

PLGA Polylactide-co-glyoclide

RPE Retinal pigment epithelial

RVP Retinal vascular permeability

STZ Streptozotocin

TIMP3 Tissue inhibitor of metalloproteinase-3

TNF-α Tumor necrosis factor-alpha

VEGF Vascular endothelial growth factor

VEGFR Vascular endothelial growth factor receptor

VPF Vascular permeability factor

The term “biocompatible” is recognized in the ad when used in reference to a material, such as the preferred polymeric composition of the present disclosure. For example, biocompatible polymers include polymers that are neither themselves toxic to the host (e.g., an animal or human), nor degrade (if the polymer degrades) at a rate that produces monomeric or oligomeric subunits or other byproducts at toxic concentrations in the host.

The term “biodegradable” is recognized in the art and includes formulations and compositions, such as the preferred polymeric composition of the disclosure, that are intended to degrade during usage. For example, biodegradable polymers include polymers that degrade, breakdown or digested (eg., by a biochemical process) into smaller subunits.

The term “encapsulated” is recognized in the art when used in reference to a material, such as a therapeutic agent, and a composition, such as the preferred polymeric composition of the disclosure. The term includes incorporating, formulating, or otherwise including the material into a composition that allows for release, such as sustained release, of the material in a desired application. Encapsulation includes any manner by which a therapeutic agent or other material is incorporated into a polymer matrix. For example, incorporation may be achieved through attachment to a monomer of the polymer (by covalent, ionic, or other binding interaction), envelopment of the agent in a coating layer of polymer, distribution throughout a polymeric matrix, encapsulation inside the polymeric matrix, etc.

The term “nanoparticle” is recognized in the art when used in reference to a structure less than about one micron in diameter. Corresponding terms recognized in the art include “microparticle,” “microsphere,” “nanosphere,” and “nanocapsule.”

The phrase “therapeutically effective amount” is recognized in the art when used in reference to an amount of the therapeutic agent that produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation.

The term “treatment” is recognized in the art and includes inhibiting or impeding the progress of a disease, disorder or condition and relieving or regressing a disease, disorder, or condition. Treatment of a disease or condition includes ameliorating at least one symptom of the particular disease or condition, even if the underlying pathophysiology is not affected, such as treating the pain of a subject by administration of an analgesic agent even though such agent does not treat the cause of the pain.

The present disclosure is concerned primarily with treatment of vascular abnormalities utilizing nanoparticles which contain vascular leakage angiogenic inhibitors or expression plasmids/vectors for vascular leakage angiogenic inhibitors. In particular, though not exclusively, the treatment is targeted towards ocular vascular abnormalities, including diabetic retinopathy, diabetic macular edema, age-related macular degeneration, sickle cell retinopathy, retinal vein occlusion, retinopathy of prematurity, other forms of retinopathy and diseases resulting from retinal neovascularization, retinal inflammation or retinal vascular leakage, and other vascular abnormality-related diseases, including cancer and arthritis.

In accordance with one aspect of the present disclosure, a method is provided for treating vascular abnormalities in a patient. The method comprises administering to the patient a therapeutically effective amount of an agent that reduces vascular leakage and angiogenesis. The agent is provided as biocompatible nanoparticles that contain an vascular leakage angiogenic inhibitor and/or an vascular leakage angiogenic inhibitor expression plasmid/vector.

In one embodiment, the vascular abnormality is located in the retina and other sites and can be neovascularization and/or vascular leakage.

The method for treating vascular abnormalities includes a biocompatible nanoparticle that is a polymer. In one embodiment, the polymer is PLGA.

The method for treating vascular abnormalities includes an vascular leakage angiogenic inhibitor and/or an vascular leakage angiogenic inhibitor expression plasmid/vector for an endogenous human peptide. In one embodiment, the vascular leakage angiogenic inhibitor is kringle 5.

In the preferred embodiment, the method for treating vascular abnormalities is a sustained therapeutic effect.

In accordance with another aspect of the present disclosure, a method is provided for preparing an agent for treating vascular abnormalities. The method comprises encapsulating an vascular leakage angiogenic inhibitor and/or vascular leakage angiogenic inhibitor expression plasmid/vector in biocompatible nanoparticles.

The method for preparing an agent for treating vascular abnormalities includes a biocompatible nanoparticle that is a polymer. In one embodiment, the polymer is PLGA.

The method for preparing an agent for treating vascular abnormalities includes an vascular leakage angiogenic inhibitor and/or an vascular leakage angiogenic inhibitor expression plasmid/vector for an endogenous human or mammalian peptide. In one embodiment, the vascular leakage angiogenic inhibitor is kringle 5.

In one embodiment, encapsulating an vascular leakage angiogenic inhibitor and/or vascular leakage angiogenic inhibitor expression plasmid/vector comprises dissolving the polymer in a solvent, mixing the vascular leakage angiogenic inhibitor or vascular leakage angiogenic inhibitor expression plasmid/vector with the polymer, emulsifying the mixture, and evaporating the solvent.

In accordance with a further aspect of the present disclosure, an agent is provided for treating vascular abnormalities. The agent comprises biocompatible nanoparticles containing an vascular leakage angiogenic inhibitor and/or vascular leakage angiogenic inhibitor expression plasmid/vector.

In one embodiment, the vascular abnormality is located in the retina and can be retinal neovascularization and/or retinal vascular leakage.

The agent for treating vascular abnormalities includes a biocompatible nanoparticle that is a polymer. In one embodiment, the polymer is PLGA.

The agent for treating vascular abnormalities includes an vascular leakage angiogenic inhibitor and/or an vascular leakage angiogenic inhibitor expression plasmid/vector for an endogenous human peptide. In one embodiment, the vascular leakage angiogenic inhibitor is kringle 5.

In one embodiment, intraocular, periocular, topical, or systemic administration of the nanoparticles containing vascular leakage angiogenic inhibitors provide transient treatment for vascular abnormalities. In the preferred embodiment, the usage of expression plasmids/vectors for vascular leakage angiogenic inhibitors allows for a sustained delivery of the vascular leakage angiogenic inhibitor for long-term treatment.

In one aspect of the disclosure, nanoparticles are used for delivering molecules or plasmids/vectors to treat vascular abnormalities. The nanoparticles are sized such for reducing obstruction in delivery to the target location while protecting the encapsulated material from undesired degradation and digestion. Preferably, the nanoparticles are sized to easily penetrate the cell membrane, including but not limited to a size of 30-500 nanometers in diameter. In addition to intracellular delivery of the material, it also possible that the nanoparticles are sized such to undergo endocytosis, thereby obtaining access to the cell. In other instances, the nanoparticles may not undergo endocytosis and remain extracellular so that the extracellular degradation of the nanoparticles will release the therapeutic agent so that if may act on a cell surface receptor to induce the effect of inhibiting vascular leakage and angiogenesis.

The nanoparticles are formulated from a chemical or biological polymer or biological material such as proteins, with the option of incorporating other compounds, such as chitosan, albumin, poly(ethylene-imine) (PEI), or additional polymers. The selection of polymer is based on desired sustained release characteristics, safety, biocompatibility, biodegradability, and ability to protect DNA from degradation in lysosomes. The nanoparticles may also contain a peptide or chemical molecule on the outer shell to facilitate receptor-mediated cellular endocytosis to certain cells. In the preferred embodiment, poly D, L-lactide-co-glycolide (PLGA) is used to formulate nanoparticles, though the polymer may be selected from a group which includes poly(ethylene-co-vinyl acetate) (EVAc), poly(ethylene-glycol), and copolymers of poly(ethylene-oxide) with poly(L-lactic acid) or with poly(beta-benzyl-L-aspartate).

Other examples of biodegradable polymers that may be used include polyesters, such as poly(caprolactone), poly(glycolic acid), poly(lactic acid), and poly(hydroxybutryate); polyanhydrides, such as poly(adipic anhydride) and poly(maleic anhydride); polydioxanone; polyamines; polyamides; polyurethanes; polyesteramides; polyorthoesters; polyacetals; polyketals; polycarbonates; polyorthocarbonates; polyphosphazenes; poly(malic acid): poly(amino acids); polyvinylpyrrolidone; poly(methyl vinyl ether); poly(alkylene oxalate); poly(alkylene succinate); polyhydroxycellulose; chitin; chitosan; and copolymers and mixtures thereof.

The nanoparticles may be formed by a wide variety of techniques including, phase separation by emulsification, diffusion, and subsequent organic solvent evaporation, coacervation-phase separation, melt dispersion, interfacial deposition, in situ polymerization, spray drying and spray congealing, air suspension coating, supercritical carbon dioxide (CO₂), and pan and spray coating. Though generally spherically shaped, the nanoparticles may be fabricated using known techniques into other desired shapes, such as a tubular form.

In another aspect of the disclosure, vascular leakage angiogenic inhibitors are used for treatment of vascular abnormalities. Individual or multiple vascular leakage angiogenic inhibitors may be used and include peptides, such as K5 and endostatin, proteins, such as PEDF, angiostatin, and tissue inhibitor of metalloproteinase-3 (TIMP3), and other molecules, such as antisense oligodeoxynucleotide against VEGF. The vascular leakage angiogenic inhibitors may be natural or synthetic, human-derived or animal-derived. In a preferred embodiment, the vascular leakage angiogenic inhibitor is plasminogen kringle 5 (K5), an endogeneous human peptide. As discussed earlier and in later examples, K5 has been shown to inhibit retinal neovascularization and reduce vascular leakage.

In a different aspect of the disclosure, expression plasmids/vectors for vascular leakage angiogenic inhibitors are used for sustained treatment of vascular abnormalities. The expression plasmids/vectors allow for nanoparticle-mediated gene delivery of vascular leakage angiogenic inhibitors. Polynucleotides, which include genomic DMA, genomic RNA, cDNA and mRNA (double stranded, as well as sense and antisense strands), are provided which encode the polypeptides of an vascular leakage angiogenic inhibitor. The polynucleotides can be synthesized chemically, or isolated by various approaches including polymerase chain reaction (PCR), ligase chain reaction (LCR), and cloning from a genomic or cDNA library.

The expression vectors generally include a promoter, an origin of replication, a translation initiation site, and may optionally include a signal peptide (secretion signal!) a poly-adenylation site, an internal ribosome entry site (IRES) and a translational termination site. Preferably the expression vectors are non-viral, such as plasmids and cosmids and cell penetration peptides, though viral DNA elements may also be used. The coding sequence may also contain a signal peptide or leader sequence for secretion of the polypeptide out of the host cell. Many commercial expression vectors may be used, including pcDNA3.1 (Invitrogen), pMC1neo (Stratagene), pXT1 (Stratagene), pSG5 (Stratagene), EBO-pSV2-neo (ATCC 37593) pBPV-1(8-2) (ATCC 37110), pdBPV-MMTneo (342-12) (ATCC 37224), pRSVgpt (ATCC 37199), pRSVneo (ATCC 37198), pSV2-dhfr (ATCC 37146), pUCTag (ATCC 37460), and IZD35 (ATCC 37565), pLXIN and pSIR (CLONTECH), pIRES-EGFP (CLONTECH). In a preferred embodiment, amplified K5 cDNA, a signal peptide, and a histidine tag sequence are cloned into a pcDNA3.1(+) expression vector, (other tag or peptide, such as flag, EGFP!)

In a further aspect of the disclosure, the treatment for vascular abnormalities includes administration of a therapeutically effective amount of nanoparticles which contain vascular leakage angiogenic inhibitors or expression vectors/plasmids for vascular leakage angiogenic inhibitors. The amount of nanoparticles and the concentration of material encapsulated by the nanoparticles is adjusted, taking one another into consideration, to provide a desired dosage based on treatment conditions. Treatment conditions include the type of therapeutic agent used, the site treated, the method of delivery, the type of patient (e.g., human or non-human, adult or child), and the nature and severity of the disease or condition.

The desired amount of nanoparticles depends on multiple considerations, including its polymer composition, rate of adsorption and degradation, size, and form of administration. The desired concentration of the material in the nanoparticle also depends on multiple considerations, including its potency, rate of release from the nanoparticle, and rate of absorption and degradation.

Administration of the nanoparticles may be achieved through various methods to different parts of the body, including intravenous, intradermal, subcutaneous, oral (e.g., inhalation), transdermal (i.e., topical), and transmucosal administration. In the preferred embodiment, intravitreal injection is used to administer K5-NP to the retina. Other forms of injection to the retina, such as subconjunctival, retrobulbar, subcutaneous, and intraperitoneal injection can also be used.

### EXAMPLES

A more complete understanding of the present invention can be obtained by reference to the following specific examples and figures. The examples and figures are described solely for purposes of illustration and are not intended to limit the scope of the disclosure. Changes in form and substitution of equivalents are contemplated as circumstances may suggest or render expedient. Although specific terms have been employed herein, such terms are intended in a descriptive sense and not for purposes of limitations. Modifications and variations of the disclosure as hereinbefore set forth can be made without departing from the spirit and scope thereof, and, therefore, only such limitations should be imposed as are indicated by the appended claims.

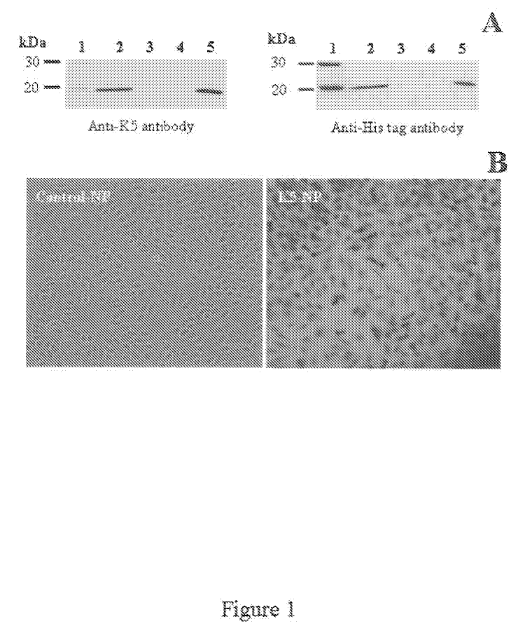

FIG. 1 shows K5 expression from K5-NP in vitro. ARPE19 cells were grown to 70% confluence in a medium containing 5% FBS. The culture medium was replaced by a serum-free medium. Control-NP and K5-NP were separately added into the medium to 1 μg/ml and incubated with the cells for 72 h. The medium was collected and concentrated using 3-K cut-off Microcentricon. Total protein concentrations in the media were measured using the Bradford assay. The same amount of total proteins (20 μg) was applied for Western blot analysis separately using a polyclonal antibody specific for human K5 and an anti-His-tag antibody. In FIG. 1A, lane 1 contained molecular weight markers, lane 2 contained medium from cells treated with K5-NP, lane 3 contained untreated cells, lane 4 contained cells treated with Control-NP, and lane 5 contained purified K5 peptide as positive control. ARPE19 cells were grown overnight on glass slides and treated with K5-NP and Control-NP at 1 μg/mL for 3 days, then washed and fixed. Shown in FIG. 1B at 400× magnification, the cells were immunostained with a monoclonal antibody against the His tag using a 3,3′-diaminobenzidine color reaction which shows brown color in positive immunostaining.

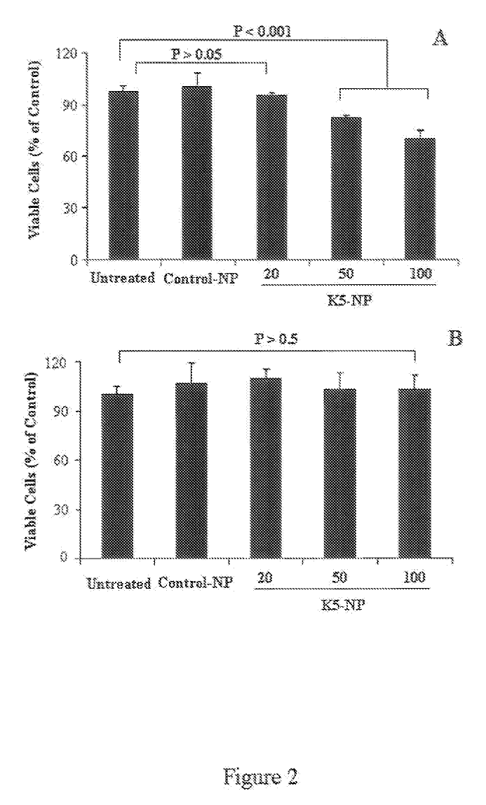

FIG. 2 shows the effect of K5-NP on ARPE19 cells and BRCEC growth. ARPE19 cells and BRCEC grown in media containing 2% of FBS were treated with K5-NP at the concentrations as indicated for 3 days. The viable cells were quantified using the MTT assay. As seen in FIG. 2A, K5-NP showed a dose-dependent inhibition on cell viability in BRCEC. FIG. 2B shows that at the same doses, K5-NP did not inhibit ARPE19 cell growth.

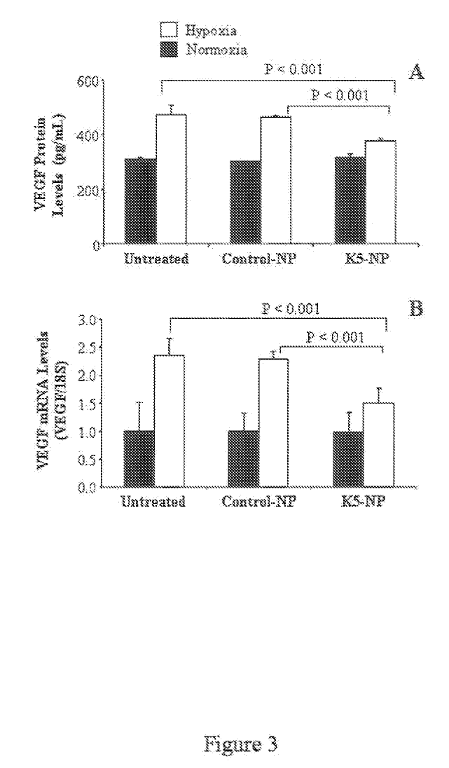

FIG. 3 shows the effect of K5-NP on VEGF over-expression under hypoxia. ARPE19 cells were treated separately with 1 μg/ml Control-NP and K5-NP for 72 h. The culture medium was replaced with serum-free medium, and the cells were exposed to hypoxia for 24 h. In FIG. 3A, VEGF secreted into the medium was measured by ELISA and normalized by total protein concentrations in the medium. In FIG. 3B, the total RNA was extracted from the treated cells, and VEGF mRNA levels were quantified by real-time RT-PCR and normalized by 18S rRNA levels. The values are mean±standard deviation (n=3).

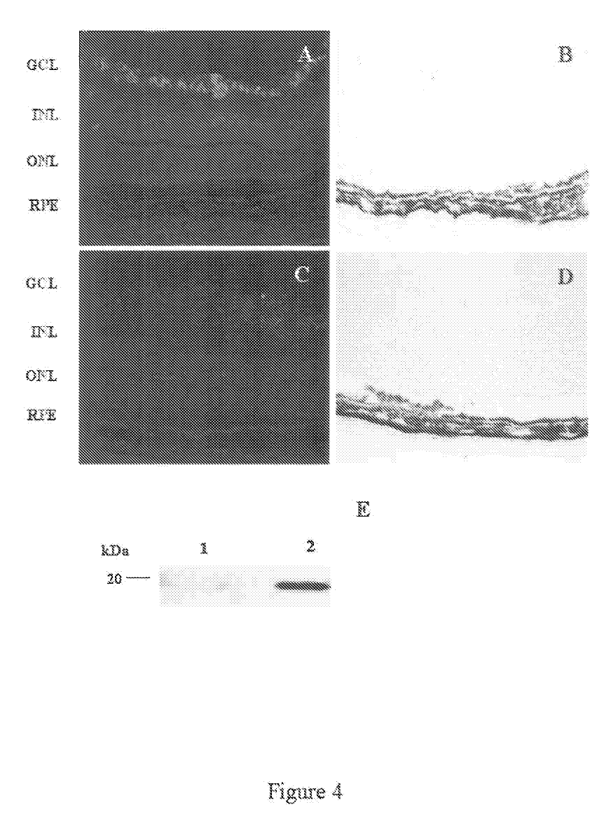

FIG. 4 shows K5 expression in the rat retina after an intravitreal injection of K5-NP. K5-NP and Control-NP were separately injected into the vitreous of OIR rats at age of P12. In FIG. 4A-4D, K5 expression was examined at age P18 by immunohistochemistry in ocular sections using an anti-His-tag antibody. FIG. 4A shows an immunostaining image from an eye injected with K5-NP and FIG. 4B shows an immunostaining image from an eye injected with Control-NP. FIGS. 4C and 4D are phase contrast images of the same areas of 4A and 4B, respectively. In FIG. 4E, K5 levels in the retina after the K5-NP injection were compared using Western blot analysis with 100 μg total proteins from each sample blotted with the anti-His tag antibody. No K5 expression was found in the eyecup treated with Control-NP (lane 1), whereas K5 expression was detected in the eyecups after the K5-NP injection (lane 2).

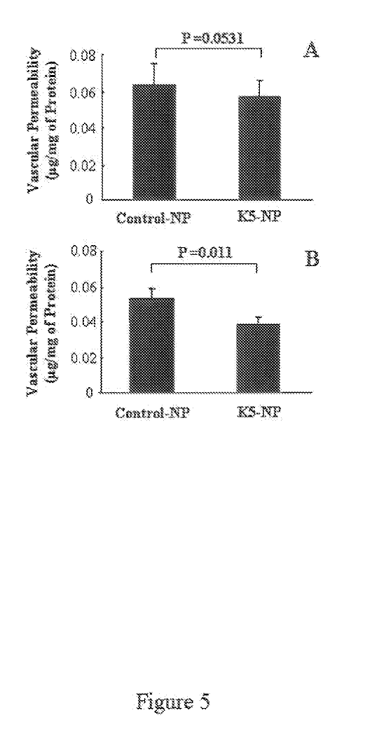

FIG. 5 shows the effect of K5-NP on retinal vascular permeability in OIR rats. OIR rats received an intravitreal injection of 2.2 μg (FIG. 5A) or 8.8 μg (FIG. 5B) of K5-NP into the right eye and the same dose of Control-NP into the left eye at age P12. Retinal vascular permeability was measured using the Evans blue-albumin leakage method at P16. The vascular leakage was normalized by total protein concentrations in the retina, averaged within the group (mean±standard deviation, n=4) and compared between contralateral eyes using paired Student's t test. FIG. 5B shows that the eyes injected with 8.8 μg/eye K5-NP showed a significantly lower vascular permeability compared, to the contralateral eye (P<0.05, n=7).

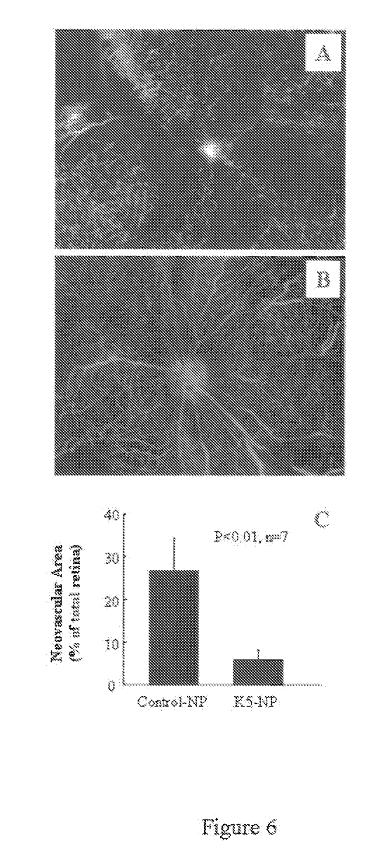

FIG. 6 shows the effect of K5-NP on retinal neovascularization in OIR rats after intravitreal injection of K5. K5-NP was infected into the vitreous of the right eyes (8.8 μg/eye) and the same amount of Control-NP into the contralateral eyes of 7 OIR rats at age P12. Retinal vasculature was examined using fluorescein angiography at P18 as described in Methods. FIG. 6A shows a representative retinal angiograph from the eyes injected with Control-NP and FIG. 6B shows a representative angiograph from the K5-NP-injected eyes (×40 magnification). Retinal neovascularization was semi-quantified by measuring the neovascular area in the retina and expressed as a percentage of the total retina area (mean±standard deviation, n=7). The difference of the neovascular area was compared between the contralateral eyes using paired Student's t test.

Experimental Methods

Construction of expression vector for K5: The human K5 cDNA (362 bp) was amplified by PCR using a pair of primers containing a 6× histidine sequence at the C-terminus of K5. To construct the eukaryotic expression and secretion vector for K5, a 52-bp linker encoding the signal peptide (SP) was cloned into a pcDNA3.1(+). The amplified K5 cDNA with the histidine tag sequence was subcloned into pcDNA3.1(+) vector at BamHI and XbaI sites, in frame with the SP sequence. The resulting pcDNA3.1(+)-SP-K5-6His expression construct was confirmed by restriction digestion and DNA sequencing.

Preparation of poly (lactide-co-glycolide) PLGA:Chitosan pK5 nanoparticles: PLGA;Chitosan nanoparticles containing pK5-plasmid were prepared using a previously reported emulsion-diffusion-evaporation technique (33) with some modifications. Briefly, 15.5 mg of PLGA (50:50, i.v. 0.17, Birmingham Polymers Inc.) was dissolved in 5 ml of ethyl acetate. A 1% w/v PVA solution was prepared and then chitosan chloride (Nova Matrix PCL 113, 2.5 mg) was dissolved under constant stirring. Plasmid solution (1 ml containing 350 □g plasmid) was modified with 10% w/v sodium sulfate and added to the chitosan solution for complexation and pDNA condensation under constant stirring. Both solutions were then combined and emulsified with a probe sonicator for 4 min at 36 W. To the emulsion, approximately 30 ml of Milli-Q H2O was added and stirred on a magnetic plate stirrer for 3 hrs to evaporate the solvent. The particle suspension was ultracentrifuged, resuspended in Milli-Q H2O and the procedure was repeated twice. Upon final resuspension in Milli-Q H2O the nanoparticle suspension was lyophilized to obtain dry particles.

Particle size and zeta-potential measurement: The particle size and size distribution were determined using dynamic light scattering (Brookhaven Instruments Corp., Holtsville, N.Y.). The same equipment was used to determine the zeta-potential of the particles.

Estimation of plasmid loading in nanoparticles: For plasmid loading estimation, the lyophilized product (0.2 mg) was dispersed in 1 mL of methylene chloride to dissolve the polymer, followed by extraction of the plasmid into 2 mL of Tris-EDTA (TE) buffer. An aliquot of the TE buffer fraction was analyzed for the absorbance at 260 nm to determine the plasmid content per mg of nanoparticles.

Western blotting: Conditioned, serum-free media were collected from the cells treated with K5-NP for 3 days, supplemented with proteinase inhibitors, and concentrated using Centricon. Protein concentrations were measured by the Bradford assay. The same amount of proteins in each sample was resolved by sodium dodecyl sulfate-polyacrylamide gel electrophoresis (SDS-PAGE) using 15% gels and electrotransferred onto nitrocellulose membranes. The membranes were blocked with 5% non-fat milk and separately blotted with a chicken antibody specific for human K5 (1:100 dilution) and a monoclonal antibody against the histidine tag (Chemicon, 1:500 dilution). After thorough washes, peroxidase-conjugated goat anti-chicken IgY (1:500) and goat anti-mouse IgG (1:500) antibodies were added, respectively, and incubated with the membrane. The signal was developed with enhanced chemiluminescence (Chemicon, Temecula, Calif.).

Immunohistochemistry: For immunocytochemical analysis, ARPE19 cells, a human RPE cell line, grown on coverslide were fixed with 4% paraformaldehyde and blocked by incubation with normal goat serum. Then the cells were immunostained with a monoclonal antibody against the His-tag overnight at 4□C, in 1% BSA in PBS. After washes, the primary antibody was detected by sequential incubations with a biotinylated anti-mouse IgG antibody and HRP conjugated streptavidin. The signal was developed with 3,3′-diaminobenzidine color reaction.

In vivo, eyes were fixed and sectioned at 5 □m in thickness, and the sections were washed in phosphate buffered saline (PBS). The sections were incubated and blocked with 20% goat serum in 0.1% Triton X-100/1% bovine serum albumin (BSA; Sigma-Aidrich, St. Louis, Mo.) in PBS. Following PBS washes, the antibody specific for the His-tag was added and incubated with the sections at 4□C overnight. The sections were rinsed several times with PBS and incubated with an FITC conjugated anti-mouse IgG antibody for 1 h. The slides were then rinsed in PBS and viewed under a fluorescent microscope.

Enzyme-linked immunosorbent assay (ELISA) of VEGF: ARPE19 cells at 70-80% confluence were treated with K5-NP or Control-NP (nanoparticles without the K5 DNA) at a final concentration of 1 □g/ml in serum-free medium. To induce hypoxia, the cells were exposed to 1% oxygen and 5% C02 for 24 h. The control cells were cultured under normoxia. The same volume of the conditioned media was used for ELISA specific for human VEGF using a VEGF ELISA kit (R&D Systems Inc. Minneapolis, Minn.) according to manufacturer's protocol.

Real-time reverse inscription-polymerase chain reaction (RT-PCR): Total RNA was isolated from ARPE19 cells using Qiagen RNeasy mini-kit (Qiagen Inc., Valencia, Calif.), according to the manufacturer's protocol. RNA integrity was examined by 1% agarose gel electrophoresis. RNA (1 μg) was reverse-transcribed at 42□C for 60 min, using reverse transcriptase (Roche Inc., Indianapolis, Ind.). The cDNA was used for real-time PCR as described previously (34). The primers used for human VEGF (5′ CAGAGCGGAGAAAGCATTTG and 3′ TGGTTCCCGAAACCCTGAGG) amplified a 180-bp fragment of VEGF. The 18S rRNA was amplified using primers (5′ TGCTGCAGTTAAAAAGCTCGT, and 3′ GGCCTGCTTTGAACA CTCTAA) for normalization of the VEGF mRNA levels. Quantification was calculated as follows: mRNA levels (percent of control)=2^{Δ}(ΔC_{T}⁾, with ΔC_{T}=C_{T,VEGF}−C_{T,18S} and Δ(ΔC_{T})=ΔC_{T,normoxia samples}−ΔC_{T,hypoxia samples} (34).

Rat model of oxygen-induced retinopathy (OIR) and intravitreal injection of K5-NP: Brown Norway rats were employed for the OIR model following an established protocol (31). Newborn rats at age of postnatal day 7 (P7) were placed in an oxygen-regulated chamber (75% O2±1.5%) for 5 days and returned to room air at P12 to induce retinal vascular leakage and neovascularization. Age-matched control rats were raised in normoxia conditions.

For injection of K5-NP, the OIR rats were anesthetized, and pupils were dilated with topical application of phenylephrine (2.5%) and tropicamide (1%), Sclerotomy was performed approximately 0.5 mm posterior to the limbus. A glass capillary connected to a syringe filled with 2 μL of nanoparticles was introduced through the sclerotomy site into the vitreous cavity, and the nanoparticles were slowly injected. All the animal experiments were performed in compliance with the ARVO Statement for the Use of Animals in Ophthalmic and Vision Research.

Retinal vascular permeability assay: Retinal vascular permeability was measured in OIR rats at P16 using the Evens blue-albumin leakage assay following an established protocol (35). Briefly, Evans blue (Sigma-Aldrich, St. Louis, Mo.), 30 mg/ml in PBS, was injected into the tail vein (10 mg/kg body weight) and allowed to circulate for 60 min. After collection of the plasma, the Evans blue in the circulation was removed by perfusion with PBS. The retinas were carefully dissected and homogenized. Evans blue in the retina and the plasma was extracted and measured with a spectrophotometer. Concentrations of Evans blue in the retina were normalized by total retinal protein concentration and by the concentration of Evans blue in the plasma.

Fluorescein retinal angiography: Retinal vasculature was visualized by fluorescein angiography as described (12, 31). Briefly, high molecular weight fluorescein-conjugated dextran (Mw:2000 kDa, Sigma-Aldrich, St. Louis, Mo.) was injected into the left ventricle. The eyes were enucleated and immediately fixed in 4% paraformaldehyde (Sigma-Aldrich, St. Louis, Mo.). The retinas were flat-mounted on slides and imaged by a fluorescent microscope. The neovascular area was measured in the retina using SPOT software. The statistic difference was tested using Student's t test.

Experimental Results

Herein, an expression plasmid of K5 was encapsulated with PLGA polymer to form nanoparticles and tested for the ability of these K5-nanoparticles (K5-NP) to reduce ischemia-induced retinal vascular leakage and retinal neovascularization in an animal model.

Characteristics of K5-NP. Nanoparticle size, polydispersity index, zeta-potential, and plasmid loading were measured with four different nanoparticle batches and the mean±SD for the various parameters is shown below. Nanoparticle formulations exhibited a mean hydrodynamic diameter of 260±30 nm, a polydispersity index of 0.28±0.005, zeta-potential of 8.4±2.4 mV, and a plasmid loading of 11±1.4 μg/mg particles.

K5 expression from K5-NP in cultured cells, ARPE19 cells were used as an in vitro model, as it expresses high levels of VEGF, a major factor inducing angiogenesis and vascular permeability (5). ARPE19 cells were transfected with K5-NP or Control-NP. The concentrated conditioned serum-free media from the transfected ARPE19 cells were blotted separately with the anti-K5 and anti-His-tag antibodies (FIG. 1A). Both of the antibodies detected significant amounts of K5 with the expected molecular weight secreted from the cells transfected with K5-NP, but not from the cells transfected with Control-NP.

The K5 expression was also determined by immunocytochemistry using the anti-His tag antibody. The antibody detected K5 signal in most transfected cells but not in cells transfected with Control-NP (FIG. 1B).

Effect of K5-NP on endothelial cell growth. It has been documented that K5peptide specifically inhibits endothelial cell growth (11). To determine the effect of K5-NP, primary bovine retinal capillary endothelial cells (BRCEC) and ARPE19 cells were treated with K5-NP for 3 days and viable cells were quantified using the MTT assay. As shown in FIG. 2, K5-NP induced a dose-dependent reduction in BRCEC numbers, when compared to the untreated cells and the cells treated with Control-NP. In contrast, the same K5-NP treatment did not result in significant reduction in ARPE19 cells (FIG. 2), suggesting that the inhibitory effect of K5-NP on cell growth is endothelial cell-specific.

Down-regulation of VEGF expression by K5. Our previous studies showed that K5 inhibits angiogenesis via down-regulation of VEGF expression (23). Here we evaluated the effect of K5-NP on VEGF expression by measuring VEGF secretion and VEGF mRNA levels. As shown by ELISA, ARPE19 cells over-expressed VEGF after the exposure to hypoxia (1% O₂) for 24 h (FIG. 3). K5-NP treatment significantly reduced the VEGF secretion induced by hypoxia, to a level similar to that from the cells under normoxia (FIG. 3A). Similarly, real-time RT-PCR showed that K5-NP significantly down-regulated the VEGF mRNA expression under hypoxia, while Control-NP did not affect VEGF expression under the same conditions, suggesting that K5-NP-mediated VEGF down-regulation occurs at the mRNA level (FIG. 3B).

Expression and localization of K5 in the retina after an intravitreal injection of K5-NP. To examine the expression of K5 from K5-NP in the retina, 8.8 μg K5-NP was injected into the vitreous of the right eye and the same amount of Control-NP into the left vitreous of the OIR rats at age of P12, after exposure to 75% oxygen. At age of P18, the immunohistochemistry was performed on retina sections using the anti-His-tag antibody. The antibody defected high levels of K5 expression in the inner retina, mainly in the ganglion cell layer in the eyes injected with the K5-NP but not in the contralateral control eyes. Western blot analysis using the anti-His-tag antibody detected a single K5 band with an expected molecular weight in the retinas injected with K5-NP but not in the control retinas from the contralateral eyes (FIG. 4E).

K5-NP reduces retinal vascular leakage in OIR rats. Our previous studies showed that OIR rats develop significant retinal vascular leakage or the blood retinal barrier (BRB) breakdown at age of P16, 4 days after the exposure to 75% oxygen (13, 24). To evaluate the effect of K5-NP on retinal vascular leakage, K5-NP was intravitreally injected into the right eye (2.2 and 8.8 μg/eye) at age P12 and the same amount of Control-NP into the contralateral eye. The vascular leakage was measured at P16 using the Evans blue-albumin leakage method and compared between the contralateral eyes. The results showed that the eyes injected with 8.8 μg of K5-NP had significantly lower vascular leakage than that in the contralateral eyes injected with the same dose of Control-NP (P=0.011, n=7). In contrast, the injection of 2.2 μg of K5-NP did not result in statistically significant decrease of vascular leakage (P=0.0531, n=7) (FIG. 5).

K5-NP inhibits ischemia-induced retinal neovascularization. To evaluate the effect of K5-NP on retinal neovascularization, 8.8 μg of K5 NP was injected intravitreally into the right eye of the OIR rats at P12 and Control-NP into the left eye. The retinal vasculature was visualized and examined by fluorescein retinal angiography. Retinal angiography on retinal flat mounts showed that the eyes injected with Control-NP developed severe retinal neovascularization in the OIR rats (FIG. 6). In contrast, a single K5-NP injection ameliorated the retinal neovascularization in the same rats by decreasing neovascular areas and non-perfusion areas in the retina. The neovascularization was semi-quantified by measuring the ratio of the neovascular area to the total retina area, which showed that the eyes injected with K5-NP have significantly decreased retinal neovascular area in the OIR rats, compared to those injected with Control-NP (FIG. 6).

Retinal vascular leakage, the direct cause of macular edema, and retinal neovascularization are the major causes of vision loss in diabetic patients (1). Sustained reduction of vascular leakage and suppression of pathogenic retinal neovascularization are the goals for the treatment of diabetic retinopathy. Although previous studies showed that peptide angiogenic factors such as K5, angiostatin, etc. effectively reduce vascular leakage and inhibit retinal neovascularization in animal models (13, 25), the difficulty in achieving sustained ocular delivery represents a major hurdle for their therapeutic applications. The present study represents the first approach to combine an endogenous vascular leakage angiogenic inhibitor with nanotechnology in the treatment of diabetic retinopathy. Our results showed that an intravitreal injection of K5-NP mediated high levels of K5 expression in the retina and induced significant reduction of retinal vascular leakage. Further, K5-NP effectively blocked ischemia-induced retinal neovascularization. This study revealed therapeutic potential of nanoparticle-mediated gene delivery of vascular leakage angiogenic inhibitors in the treatment of diabetic retinopathy.

Nanoparticles formulated using PLGA polymers are recently being investigated as a new gene delivery system because of their sustained release characteristics, biocompatibility, biodegradability and ability to protect DNA from degradation in lysosomes (26, 27). Our immunoblotting results showed that K5-NP mediated high-level K5 expression both in vitro and in vivo, suggesting that K5-NP is efficiently internalized and released into the cytoplasmic compartment rather than being retained in the degradative lysosomal compartment. Moreover, the expressed K5 is secreted into the extracellular space and has biological activity of inhibiting endothelial cell growth and VEGF over-expression under hypoxia.

Cytotoxicity is a potential concern in some nanoparticles-mediated gene deliveries. We have determined potential cytotoxicity in cultured ARP19 cells and in the retina. In ARPE19 cells treated with K5-NP at high concentrations, no significant reduction in cell viability was observed. In contrast, the same concentrations of K5-NP significantly inhibited endothelial cell growth, suggesting an endothelial cell-specific inhibitory effect of K5. After an intravitreal injection of K5-NP, no significant alterations were observed in the retinal histology (data not shown), suggesting that PLGA NP of K5 has no severe toxicities at the dose used.

Our previous study demonstrated that K5 down-regulates the expression of VEGF through inhibiting the activation of HIF-1 and p42/p44 MAP kinase (23). As VEGF is a major permeability and angiogenic factor, down-regulation of VEGF expression is believed to be responsible for the effects of K5 on vascular leakage and retinal neovascularization (13, 14). To confirm whether the expressed K5 from nanoparticles plays role in blocking the VEGF over-expression under hypoxia, ELISA and quantitative RT-PCR were performed in ARPE19 cells. Exposure of quiescent ARPE19 cells to hypoxia increased secretion of VEGF and the VEGF mRNA levels. The over-expression of VEGF under hypoxia can be effectively blocked by K5-NP, suggesting that the mechanism underlying the vascular activities of K5-NP is identical to that of K5 peptide.

The OIR model is commonly used for studies of diabetic retinopathy (28-30). Although it is not a diabetic model, the pathology in the retina in this model is similar to that in diabetic retinopathy in humans (31). Moreover, VEGF over-expression has been shown to be the key pathogenic factor for retinal vascular leakage and neovascularization in this model, a pathogenic mechanism similar to that in diabetic retinopathy (7, 32). Our results showed that K5-NP reduced retinal vascular permeability in a dose-dependent manner, demonstrating a beneficial effect on vascular leakage induced by hypoxia.

To evaluate the effect of K5-NP on retinal neovascularization in the OIR model, we used fluorescein angiography on flat-mounted retinas. The results showed that a single injection of K5-NP significantly reduced the neovascular area and non-perfusion area, which have been shown to correlate with severity of diabetic retinopathy (28, 31). This result suggests that K5-NP also blocks ischemia-induced retinal neovascularization. A limitation of the OIR model, however, is that both the vascular leakage and retinal neovascularization are transient, with peaks at P16 and P18, respectively (24, 31). Therefore, the prolonged effect of K5-NP remains to be determined in other models.

In conclusion, these studies demonstrate that PLGA nanoparticles can be used for gene delivery of vascular leakage angiogenic inhibitors and thus, may be useful for research and potentially for future gene therapy for diabetic macular edema and retinal neovascularization associated with several ocular disorders such as diabetic retinopathy.

### REFERENCES CITED BY NUMERALS

1.Aiello, L. P., Gardner, T. W., King, G. L, Blankenship, G., Cavallerano, J. D., Ferris, F. L, 3rd, and Klein, R. 1998. Diabetic retinopathy. Diabetes Care 21:143-156.

2.Gardner, T. W., Antonetti, D. A, Barber, A. J., LaNoue, K. F., and Levison, S. W. 2002. Diabetic retinopathy: more than meets the eye. Surv Ophthalmol 47 Suppl 2:S253-262.

3. Miller, J. W. 1997. Vascular endothelial growth factor and ocular neovascularization. Am J Pathol 151:13-23.

4. Smith, L. E. 2003. Pathogenesis of retinopathy of prematurity. Semin Neonatol 8:469-473.

5. Aiello, L. P., Bursell, S. E., Clermont, A., Duh, E., Ishli, H., Takagi, C, Mori, F., Ciulla, T. A., Ways, K., Jirousek, M., et al. 1997. Vascular endothelial growth factor-induced retinal permeability is mediated by protein kinase C in vivo and suppressed by an orally effective beta-isoform-selective inhibitor. Diabetes 48;1473-1480.

6. Aiello, L. P., and Wong, J. S. 2000. Role of vascular endothelial growth factor in diabetic vascular complications. Kidney Inf Suppl 77:S113-119.

7. Pierce, E. A., Avery, R. L., Foley, E. D., Aiello, L. P., and Smith, L. E. 1995. Vascular endothelial growth factor/vascular permeability factor expression in a mouse model of retinal neovascularization. Proc Natl Acad Sci USA 92:905-909.

8. Gao, G., Li, Y., Zhang, D., Gee, S., Crosson, C., and Ma, J. 2001. Unbalanced expression of VEGF and PEDF in ischemia-induced retinal neovascularization. FEBS Lett 489:270-276.

9. Bouck, N. 2002. PEDF:anti-angiogenic guardian of ocular function. Trends Mol Med 8:330-334.

10. O'Reilly, M. S., Holmgren, L., Shing, Y., Chen, C., Rosenthal, R. A., Moses, M., Lane, W. S., Cao, Y., Sage, E. H., and Folkman, J. 1994. Angiostatin: a novel angiogenesis inhibitor that mediates the suppression of metastases by a Lewis lung carcinoma. Cell 79:315-328.

11. Cao, Y., Chen, A., An, S. S., Ji, R. W., Davidson, D., and Llinas, M. 1997. Kringle 5 of plasminogen is a novel inhibitor of endothelial cell growth. J Biol Chem 272:22924-22928.

12. Zhang, D., Kaufman, P. L., Gao, G., Saunders, R. A., and Ma, J. X. 2001.Intravitreal injection of plasminogen kringle 5, an endogenous vascular leakage angiogenic inhibitor, arrests retinal neovascularization in rats. Diabetologia 44:757-765.

13. Zhang, S. X., Sima, J., Shao, C., Fant J., Chen, Y., Rohrer, B., Gao, G., and Ma, J. X. 2004. Plasminogen kringle 5 reduces vascular leakage in the retina in rat models of oxygen-induced retinopathy and diabetes. Diabetologia 47:124-131.

14. Zhang, S. X., Sima, J., Wang, J. J., Shao, C., Fant, J., and Ma, J. X. 2005. Systemic and periocular deliveries of plasminogen kringle 5 reduce vascular leakage in rat models of oxygen-induced retinopathy and diabetes. Curr Eye Res 30:681-689.

15. Kootstra, N. A., and Verma, I. M. 2003. Gene therapy with viral vectors. Anna Rev Pharmacol Toxicol 43:413-439.

16. Acland, G. M., Aguirre, G. D., Ray, J., Zhang, Q., Aleman, T. S., Cideciyan, A. V., Pearce-Kelling, S. E., Anand, V., Zeng, Y., Maguire, A. M., et al. 2001. Gene therapy restores vision in a canine model of childhood blindness. Nat Genet 28:92-95.

17. Monahan, P. E., Jooss, K., and Sands, M. S. 2002. Safety of adeno-associated virus gene therapy vectors: a current evaluation. Expert Opin Drug Saf 1:78-91.

18. Tenenbaum, L., Lehtonen, E., and Monahan, P. E. 2003. Evaluation of risks related to the use of adeno-associated virus-based vectors. Curr Gene Ther 3:545-585.

19. Vijayanathan, V., Thomas, T., and Thomas, T. J. 2002. DNA nanoparticles and development of DNA delivery vehicles for gene therapy. Biochemistry 41:14085-14094.

20. Farokhzad, O. C., Cheng, J., Teply, B. A., Sheriff I., Jon, S., Kantoff, P. W., Richie, J. P., and Langer, R. 2006. Targeted nanoparticle-aptamer bioconjugates for cancer chemotherapy in vivo. Proc Natl Acad Sci USA 103:6315-6320.

21. Keegan, M. E., and Saltzman, W. M. 2006. Surface-modified biodegradable microspheres for DMA vaccine delivery. Methods Mol Med 127:107-113.

22. Luo, Q., and Saltzman, W. M. 2000. Synthetic DNA delivery systems. Nat Biotechnol 18:33-37.

23. Gao, G., Li, Y., Gee, S., Dudley, A., Fant, J., Crosson, C., and Ma, J. X. 2002. Down-regulation of vascular endothelial growth factor and up-regulation of pigment epithelium-derived factor: a possible mechanism for the anti-angiogenic activity of plasminogen kringle 5. J Biol Chem 277:9492-9497.

24. Zhang, S. X., Ma, J. X., Sima, J., Chen, Y., Hu, M. S., Ottlecz, A., and Lambrou, G. N. 2005. Genetic difference in susceptibility to the blood-retina barrier breakdown in diabetes and oxygen-induced retinopathy. Am J Pathol 166:313-321.

25. Sima, J., Zhang, S. X., Shao, C., Fant, J., and Ma, J. X. 2004. The effect of angiostatin on vascular leakage and VEGF expression in rat retina. FEBS Lett 584:19-23.

26. Cohen, H., Levy, R. J., Gao, J., Fishbein, I., Kousaev, V., Sosnowski, S., Slomkowski, S., and Golomb, G. 2000. Sustained delivery and expression of DNA encapsulated in polymeric nanoparticles. Gene Ther 7:1898-1905.

27. Panyam, J., Zhou, W. Z., Prabha, S., Sahoo, S. K., and Labhasetwar, V. 2002. Rapid endo-lysosomal escape of poly(DL-lactide-co-glycolide) nanoparticles: implications for drug and gene delivery. Faseb J 16:1217-1226.

28. Penn, J. S., Tolman, B. L., and Henry, M. M. 1984. Oxygen-induced retinopathy in the rat: relationship of retinal nonperfusion to subsequent neovascularization. Invest Ophthalmol Vis Sci 35:3429-3435.

29. Smith, L. E., Shen, W., Perruzzi, C., Soker, S., Kinose, F., Xu, X., Robinson, G., Driver, S., Bischoff, J., Zhang, B., et al. 1999. Regulation of vascular endothelial growth factor-dependent retinal neovascularization by insulin-like growth factor-1 receptor. Nat Med 5:1390-1395.

30. Spranger, J., Osterhoff, M., Reimann, M., Mohlig, M., Ristow, M., Francis, M. K., Cristofalo, V., Hammes, H. P., Smith, G., Boulton, M., et al. 2001. Loss of the antiangiogenic pigment epithelium-derived factor in patients with angiogenic eye disease. Diabetes 50:2641-2645.

31. Smith, L. E., Wesolowski, E., McLellan, A., Kostyk, S. K., D'Amato, R., Sullivan, R., and D'Amore, P. A. 1994. Oxygen-induced retinopathy in the mouse. Invest Ophthalmol Vis Sci 35:101-111.

32. Antonetti, D. A., Barber, A. J., Hollinger, L. A., Wolpert, E. B., and Gardner, T. W. 1999. Vascular endothelial growth factor induces rapid phosphorylation of tight Junction proteins occludin and zonula occluden 1. A potential mechanism for vascular permeability in diabetic retinopathy and tumors. J Biol Chem 274:23463-23467.

33. Kumar, M. N., Mohapatra, S. S., Kong, X., Jena, P. K., Bakowsky, U., and Lehr, C. M., 2004. Cationic poly(lactide-co-glycolide) nanoparticles as efficient in vivo gene transfection agents. J Nanosci Nanotechnol 4:990-994.

34. Wang, J. J., Zhang, S. X., Lu, K., Chen, Y., Mott, R., Sato, S., and Ma, J. X. 2005. Decreased expression of pigment epithelium-derived factor is involved in the pathogenesis of diabetic nephropathy. Diabetes 54:243-250.

35. Xu, Q., Qaum, T., and Adamis, A. P. 2001. Sensitive blood-retinal barrier breakdown quantitation using Evans blue. Invest Ophthalmol Vis Sci 42:789-794.

While the method and agent have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. A method of treating vascular abnormalities in a patient, said method comprising administering to the patient a therapeutically effective amount of an agent that reduces angiogenesis, said agent further comprising biocompatible nanoparticles containing at least one of an vascular leakage angiogenic inhibitor and vascular leakage angiogenic inhibitor expression plasmid/vector.

2. The method of claim 1, wherein the vascular abnormality is located in the retina.

3. The method of claim 2, wherein the vascular abnormality comprises at least one of retinal neovascularization and retinal vascular leakage.

4. The method of claim 1, wherein the biocompatible nanoparticle is a polymer.

5. The method of claim 4, wherein the polymer is PLGA.

6. The method of claim 1, wherein the vascular leakage angiogenic inhibitor is an endogenous human or mammalian peptide.

7. The method of claim 6, wherein the vascular leakage angiogenic inhibitor is kringle 5 or a biologically active fragment or variant thereof.

8. The method of claim 1, wherein the method further comprises a sustained therapeutic effect.

9. A method of preparing an agent for treatment of vascular abnormalities, said method comprising encapsulating at least one of an vascular leakage angiogenic inhibitor and vascular leakage angiogenic inhibitor expression plasmid/vector in biocompatible nanoparticles.

10. The method of claim 9, wherein the biocompatible nanoparticle is a polymer.

11. The method of claim 10, wherein the polymer is PLGA.

12. The method of claim 9, wherein the vascular leakage angiogenic inhibitor is an endogenous human or mammalian peptide.

13. The method of claim 12, wherein the vascular leakage angiogenic inhibitor is kringle 5 or a biologically active fragment or variant thereof.

14. The method of claim 10, wherein encapsulating at least one of an vascular leakage angiogenic inhibitor and vascular leakage angiogenic inhibitor expression plasmid/vector comprises dissolving the polymer in a solvent, mixing the at least one of an vascular leakage angiogenic inhibitor and vascular leakage angiogenic inhibitor expression plasmid/vector with the polymer, emulsifying the mixture, and evaporating the solvent.

15. An agent for treatment of vascular abnormalities comprising biocompatible nanoparticles containing at least one of an vascular leakage angiogenic inhibitor and vascular leakage angiogenic inhibitor expression plasmid/vector.

16. The method of claim 15, wherein the vascular abnormality is located in the retina.

17. The method of claim 16, wherein the vascular abnormality comprises at least one of retinal neovascularization and retinal vascular leakage.

18. The method of claim 15, wherein the biocompatible nanoparticle is a polymer.

19. The method of claim 18, wherein the polymer is PLGA.

20. The method of claim 15, wherein the vascular leakage angiogenic inhibitor is an endogenous human peptide.

21. The method of claim 20, wherein the vascular leakage angiogenic inhibitor is kringle 5 or a biologically active fragment or variant thereof.
